(19) United States
(12) Patent Application Publication
(10) Pub. No.: US 2004/0048925 A1
(43) Pub. Date: Mar. 11, 2004

(54) Composition and method for enhancing the bioavailability of calcium and magnesium in dietary supplements and food additives

(76) Inventors: David B. Wiley, Warsaw, OH (US); Thomas A. Dobbins, Howard, OH (US)

Correspondence Address: Thompson Hine LLP, 2000 Courthouse Plaza N.E., P.O. Box 8801, Dayton, OH, 45401-8801, US

(21) Appl. No.: 10/658,075

(22) Filed: Sep. 9, 2003

Related U.S. Application Data
(60) Provisional application No. 60/409,151, filed on Sep. 9, 2002.

(51) Int. Cl. A61K 31/19

(52) U.S. Cl. 514/557

(57) ABSTRACT

Dietary mineral supplements comprising the calcium and/or magnesium salts of 3-hydroxy3-methylbutyric acid are disclosed as efficient means of orally administering calcium and/or magnesium in order to prevent or treat calcium and magnesium deficiency pathologies. The conjoint bioavailability of these important minerals is thereby enhanced.

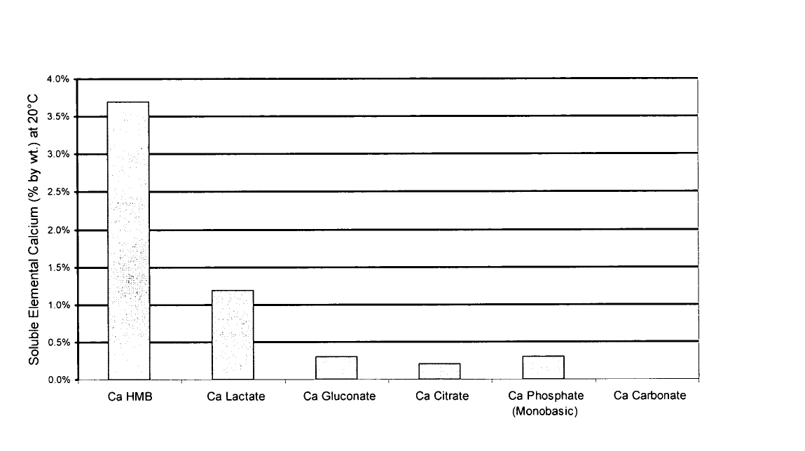

### REFERENCE TO RELATED APPLICATION

This application claims priority to U.S. provisional application Ser. No. 60/409,151, filed on Sep. 9, 2002.

### FIELD OF THE INVENTION

The present invention relates to compositions and methods for preventing and/or treating conditions associated with calcium and/or magnesium deficiencies in humans or other animals. In particular, the invention relates to compositions comprising calcium and/or magnesium salts of 3-hydroxy-3-methylbutyric acid in a form suitable for administration to a subject in need thereof.

### BACKGROUND OF THE INVENTION

In human and animal metabolism, calcium is essential for the maintenance of the functional integrity of the nervous, muscular, and skeletal systems, and cell membrane and capillary permeability. The calcium cation is an important activator in many enzymatic reactions and is essential to a number of physiologic processes including the transmission of nerve impulses; contraction of cardiac, smooth, and skeletal muscles; renal function; respiration; and blood coagulation. Calcium also plays a regulatory role in the release and storage of neurotransmitters and hormones, in the uptake and binding of amino acids, in vitamin B12 absorption, and in gastrin secretion.

The calcium of bone is in a constant exchange with the calcium of plasma. Since the metabolic functions of calcium are essential for life, when there is a disturbance in the calcium balance because of dietary deficiency or other causes, the stores of calcium in the skeletal system may be depleted to fill the body's more acute needs. Therefore, on a chronic basis, normal mineralization of bone depends upon adequate amounts of total body calcium.

Dietary calcium is absorbed through the small intestine. Approximately one third of ingested calcium is absorbed, although this fraction can vary depending upon dietary factors and the state of the small intestine. Following absorption, calcium first enters the extracellular fluid and is then rapidly incorporated into skeletal tissue.

Calcium can be obtained from a variety of dietary sources. Primary sources of calcium are dairy products, in particular milk, which account for 75% of the daily calcium intake. Foods other than dairy products generally contribute less than 200 mg of calcium daily. However, beginning in young adulthood and continuing through later life, the general population rarely consumes milk in sufficient quantities to obtain the recommended dietary levels of calcium. This diminished consumption can be caused by lactose intolerance as well as by the unattractiveness of milk as a drink for social occasions.

Vitamin and mineral supplementation is important primarily for those who have inadequate diets, including growing children. Older adults have an additional need for calcium to help prevent age-related bone loss. Postmenopausal women need additional calcium due to hormonal changes which can accelerate the rate of bone loss leading to a further diminishment in bone mass. Therefore, supplementation of the diet with a highly bioavailable source of calcium is desirable.

Osteoporosis is a condition in which an affected person's bones become increasingly porous, brittle, and subject to fracture, owing to loss of calcium and other mineral components. It is a common affliction in older persons, particularly postmenopausal women. Bone loss may also be associated with a variety of other conditions, including those brought on by long-term steroid therapy and certain endocrine disorders. If not countered, osteoporosis or bone loss may lead to fractures of the spine, hip, and long bones. U.S. Department of Agriculture consumption data suggest that 90% of adult women and 70% of adult men and teenagers do not meet the recommended daily intake (RDI) of calcium from dietary sources.

The prevention and treatment of osteoporosis and systemic hypocalcemia generally requires the oral administration of calcium supplements to increase the amount of calcium available from dietary sources for intestinal absorption. The degree of absorption will depend on the calcium preparation used and on the timing of ingestion.

The calcium compounds currently employed as dietary supplements vary widely in their elemental calcium content as well as in their solubility in aqueous media; the latter factor largely determines the bioavailability of the calcium. Bioavailability is defined as the efficiency with which a natural or manufactured source of an element delivers the element to storage or supplies it to metabolically active tissue. U.S. Pat. No. 5,468,506 teaches: “It is essential for the bioavailability of calcium that the calcium salts be soluble in the stomach and intestine. This solubility aids in making calcium more readily available for absorption. Thus, the choice of calcium salts depends upon the interaction of the salts with secretions in the stomach and intestine.”

Calcium carbonate (CaCO₃; elemental calcium content of 40% by weight) is virtually insoluble in water and must react in situ with the acids occurring in the digestive tract to produce soluble calcium salts, chiefly calcium chloride (CaCl₂). Consequently, it has been established that the bioavailability of calcium when administered as calcium carbonate is relatively poor. The alkali loading that accompanies the ingestion of calcium carbonate is undesirable and may promote the formation of calcium-containing kidney stones (‘calcium nephrolithiasis’), as taught by U.S. Pat. Nos. 4,851,221 and 6,287,607. In addition, calcium carbonate is widely reported to cause constipation.

The phosphate salts of calcium employed as dietary supplements include: monobasic calcium phosphate monohydrate (calcium acid phosphate, Ca(H₂PO₄)₂.H₂O; 16% by wt. elemental calcium); dibasic calcium phosphate dihydrate (calcium monohydrogen phosphate dihydrate, CaHPO₄.2H₂O; 23% by wt. elemental calcium); and tribasic calcium phosphate (tricalcium phosphate, Ca₃(PO₄)₂; 38% by wt. elemental calcium). The monobasic phosphate salt is moderately soluble in water, but it forms acidic solutions that have a bitter taste, while the dibasic and tribasic phosphate salts are virtually insoluble in water and consequently have poor bioavailability. The phosphate salts of calcium may exacerbate hyperphosphataemia and metastatic calcification (see C. Y. C. Pak “Calcium Disorders: Hypercalcemia and Hypocalcemia” in J. P. Kokko, R. L. Tannen, eds. Fluids and Electrolytes (Philadelphia: W. B. Saunders, 1990), pp. 596-630; K. A. Hruska and J. Connoly, Primer on the Metabolic Bone Diseases and Disorders of Mineral Metabolism (Philadelphia: Lippincott-Raven, 1996), pp. 238-245).

Calcium glycerophosphate, (CaC₃H₇O₆; 19% by wt. elemental calcium), is soluble in 50 parts of water at 20° C., forming an alkaline solution. Like the simple phosphate salts, calcium glycerophosphate may exacerbate hyperphosphataemia and metastatic calcification.

Calcium citrate tetrahydrate (Ca₃(C₆H₅O₇)₂.4H₂O; 21% by wt. elemental calcium) is soluble in 1050 parts of cold water and is better absorbed than the carbonate or phosphates of calcium (see Pak et al., “Enhanced Calcium Bioavailability from a Solubilized Form of Calcium Citrate” J. of Clin. Endocrinology and Metabolism, 65, 4, (1987) 801-805 and Harvey et al., “Dose Dependency of Calcium Absorption: A Comparison of Calcium Carbonate and Calcium Citrate” J. of Bone and Mineral Res., 3, 3, (1988)253-258), but it enhances aluminum absorption and may predispose to aluminum toxicity in patients with renal insufficiency. This disadvantage is also applicable to the mixture or complex of calcium citrate and calcium malate taught by U.S. Pat. Nos. 5,314,919 and 5,468,506.

U.S. Pat. No. 6,287,607 teaches the use of potassium calcium citrates, complex salts that contain a molar ratio of potassium to calcium of either 1:1 or 4:1 (corresponding to elemental calcium contents of 15% and 7% by wt., respectively), as highly soluble compounds that offer improved bioavailability of calcium despite their modest calcium content. However, delivering significant amounts of potassium may undesirably alter the sodium-to-potassium ratio of blood electrolytes. An excess of potassium (“hyperkalemia”) is associated with cardiac arrhythmia.

U.S. Pat. No. 5,075,499 teaches the use of calcium lactate pentahydrate (2-hydroxypropanoic acid calcium salt pentahydrate, Ca(C₃H₅O₂)₂.5H₂O; 13% by wt. elemental calcium) as a calcium dietary supplement. This compound forms a saturated 9.2% by weight aqueous solution at 20° C. with a pH of 6-7 that offers good bioavailability of calcium.

Calcium gluconate (d-gluconic acid calcium salt, Ca(C₆H₁₁O₇)₂; 9% by wt. elemental calcium) is slowly soluble in 30 parts of water at 20° C., forming a saturated solution with a pH of 6-7. The solubility of calcium gluconate in water can be increased to 20% by weight or more by adding a complexing agent like boric acid.

Magnesium is important for the proper functioning of nerves and muscles. It is an essential constituent of many enzyme systems, particularly those involved with energy generation. The body's largest stores are found in the skeleton. The recommended daily intake (RDI) of magnesium ranges from 300 to 400 milligrams in most countries; 70% of the US population do not meet the RDI.

Magnesium salts are not well absorbed from the gastrointestinal tract, accounting for the fact that the most common magnesium supplement, magnesium sulfate (MgSO₄), is also employed as an osmotic laxative. Ideally, calcium and magnesium should be co-administered; an excess of calcium relative to magnesium can cause muscle stiffness and cramping.

U.S. Pat. No. 5,219,889 teaches the use of a complex potassium-magnesium salt of citric acid as a dietary supplement. This compound, tetrapotassium monomagnesium dicitrate, has a potassium-to-magnesium molar ratio of 4:1. However, delivering significant amounts of potassium may undesirably alter the sodium-to-potassium ratio of blood electrolytes. Hyperkalemia is associated with cardiac arrhythmia.

### SUMMARY OF THE INVENTION

The present invention provides calcium and magnesium compositions useful as dietary supplements and food additives and methods for administering the compositions to prevent or treat disorders associated with hypocalcemia and/or hypomagnesia. The compositions of the present invention comprise calcium and/or magnesium salts of 3-hydroxy-3-methylbutyric acid in a form suitable for administration to a subject in need thereof. Methods for treatment of hypocalcemia and/or hypomagnesia comprising administration of a therapeutically effective regimen of calcium and/or magnesium salts of 3-hydroxy-3-methylbutyric acid to a patient in need of such treatment are also provided.

More particularly, one aspect of the invention relates to a method for preventing and/or treating a condition associated with calcium and/or magnesium deficiency in a human or other animal subject. The method includes administering a safe and effective amount of calcium 3-hydroxy-3-methylbutyrate, magnesium 3-hydroxy-3-methylbutyrate or mixtures thereof to a subject. Examples of conditions treated or prevented include, but are not limited to, osteoporosis, hypertension and bone loss.

In accordance with another aspect of the present invention, a method for providing a nutritionally supplemental amount of calcium and magnesium to a human or other animal subject is described. This method includes administering a safe and effective amount of a mineral supplement comprising calcium 3-hydroxy-3-methylbutyrate and magnesium 3-hydroxy-3-methylbutyrate to a human or other animal subject.

In accordance with another aspect of the present invention a bioavailable and water-soluble composition for conjointly administering bioavailable calcium and magnesium in an oral form to a human patient or other animal in need thereof is provided. The composition includes (a) calcium in a water-soluble form, (b) magnesium in a water-soluble form, and (c) an edible organic acid component comprising 3-hydroxy-3-methylbutyric acid.

### BRIEF DESCRIPTION OF THE DRAWING

The FIGURE illustrates the amount of elemental calcium present in a saturated aqueous solution of various calcium salts at 20° C.

### DETAILED DESCRIPTION OF THE INVENTION

All documents cited are, in relevant part, incorporated herein by reference; the citation of any document is not to be construed as an admission that it is prior art with respect to the present invention.

As used herein, the term “comprising” means that the described composition or process includes the components or steps recited but is open to the inclusion of additional components or steps. The terms “consisting essentially of” and “consisting of” are embodied in the term “comprising.”

As used herein, the term “pharmaceutically acceptable” refers to a composition that is suitable for use with humans and/or animals without undue adverse side effects (such as toxicity, irritation, or allergic response) commensurate with a reasonable benefit/risk ratio. The term “safe and effective amount” as used herein refers to a quantity of a component which is sufficient to yield a desired therapeutic response without undue adverse side effects commensurate with a reasonable benefit/risk ration when used in the manner of this invention. The specific “safe and effective amount” will vary with such factors as the particular condition being treated, the physical condition of the patient, the duration of the treatment, the nature of any concurrent therapy, and the specific formulations employed.

By “nutritional” or “nutritionally-supplemental amount” herein is meant that the mineral and vitamin sources used in the practice of this invention provide a nourishing amount of magnesium and/or calcium. This is supplemental or in addition to the amount found in the average diet. This supplemental amount typically will comprise at least about 3% of the Recommended Dietary Intake (RDI) of calcium and/or magnesium. More generally, the mineral supplements disclosed herein will contain at least 10%, more typically about 50% to 300% of the Recommended Dietary Intake (RDI) per unit dose of the supplement.

The term “elemental” as used herein means of or pertaining to the element referred to. The elements involved in the present invention are primarily calcium, magnesium and other minerals beneficial for human consumption. Elemental percentage indicates the percentage of elemental calcium, magnesium, etc. present in a composition. Accordingly, the elemental percentage of calcium in calcium carbonate does not include the percentage of carbonate present.

The compositions of the present invention are soluble at high concentrations. The term “soluble” as used herein means capable of being dissolved, going into a liquid state from a solid state. The solubility of a mineral is an indicator of how bioavailable that mineral is. (See Schaafsma, G., “Bioavailability of Calcium and Magnesium,” European J. of Clinical Nutrition, 1977, “It is clear that availability for absorption requires calcium to be solubilized, either in free ionic or complexed form.”) Solubility also plays an important role in the preparation of foods and beverages containing these supplements.

The calcium salt of 3-hydroxy-3-methylbutyric acid (synonyms: beta-hydroxy-betamethylbutyric acid; 3-hydroxy-isovaleric acid) forms a monohydrate with the chemical formula Ca(C₅H₉O₃)₂.H₂O. With an elemental calcium content of 13.7% by weight, calcium hydroxymethylbutyrate (“CaHMB”) dissolves readily in water, forming a 27% by weight saturated aqueous solution at 20° C. As indicated above, a high degree of solubility corresponds to a high degree of bioavailabilty. The FIGURE graphically compares the amount of elemental calcium present in a saturated aqueous solution of CaHMB at 20° C. with the amounts present in saturated solutions of the calcium salts currently employed as dietary supplements. As shown in the figure, there is a significant increase in elemental calcium associated with CaHMB as compared to other calcium salts used in supplements. This increase in solubility corresponds to an increase in bioavailability.

The calcium salt of 3-hydroxy-3-methylbutyric acid can be prepared by reacting a slight (i.e. 5%) stoichiometric excess calcium oxide (“unslaked lime”) or calcium hydroxide (“slaked lime”) with an aqueous solution containing free 3-hydroxy-3-methylbutyric acid, preferably at an elevated temperature of 40 to 70 degrees C. Alternatively, calcium carbonate can be employed. The unreacted excess oxide, hydroxide, or carbonate can be removed by filtration and the calcium salt 3-hydroxy-3-methylbutyric acid is recovered by crystallizing, spray-drying, or freeze-drying (lyopholizing) the resulting filtrate.

The taste of calcium hydroxy-methylbutyrate (“CaHMB”) is not objectionable and can be easily masked. Moreover, not only is CaHMB non-toxic, but the hydroxymethylbutyrate anion is reputed to have a number of other beneficial physiological effects, including binding phosphates in the treatment of uremia (M. F. Sousa et al, “Calcium beta-hydroxy-beta-methylbutyrate: potential role as a phosphate binder in uremia” Nephron 72 (1996), 391-394); promoting nitrogen retention (U.S. Pat. No. 5,348,979); enhancing immune response (U.S. Pat. No. 4,992,470); reducing blood serum cholesterol (U.S. Pat. No. 5,360,613); increasing the aerobic capacity of muscle (U.S. Pat. No. 6,103,764); and even improving a human's self-perceived emotional state (U.S. Pat. No. 6,291,525).

The magnesium salt of 3-hydroxy-3-methylbutyric acid (synonyms: beta-hydroxybeta-methylbutyric acid; 3-hydroxy-isovaleric acid) has the chemical formula Mg(C₅H₉O₃)₂. With an elemental magnesium content of 9.4% by weight, magnesium hydroxymethylbutyrate monohydrate (“MgHMB”) dissolves readily in water, forming a 32% by weight saturated aqueous solution at 20° C. This is significantly higher than the solubility of magnesium lactate (one gram in 25 ml cold water), an established commercial magnesium supplement. As in the case of the calcium salt, this high degree of solubility corresponds to a high degree of bioavailabilty, as well as chemical compatibility when co-administered with CaHMB. (Magnesium sulfate produces an insoluble precipitate when added to solutions of CaHMB).

The magnesium salt of 3-hydroxy-3-methylbutyric acid can be prepared by reacting a 5% stoichiometric excess of basic magnesium carbonate (approximate composition (MgCO₃)₄.Mg(OH)₂·5H₂O, equivalent to 40 to 42% MgO) with an aqueous solution containing free 3-hydroxy-3-methylbutyric acid, preferably at an elevated temperature of 40 to 70 degrees C. Alternatively, magnesium oxide or magnesium hydroxide can be employed. The unreacted excess carbonate, oxide, or hydroxide can be removed by filtration and the magnesium salt of 3-hydroxy-3-methylbutyric acid can be recovered by crystallizing, spray-drying, or freeze-drying (lyopholizing) the resulting filtrate.

Both the calcium and the magnesium salts of 3-hydroxy-3-methylbutyric acid are stable powders with long shelf lives. If pure, they dissolve in water to form aqueous solutions of nearly neutral or slightly alkaline pH. They are highly soluble over a broad range of pH values.

Various oral dosage forms of calcium and magnesium hydroxy-3-methylbutyrates may be used in the present invention. The calcium and the magnesium salts of 3-hydroxy-3-methylbutyric acid may be in unit dosage forms or in multiple dosage form. Such dosage forms comprise a safe and effective amount of the calcium and/or magnesium hydroxy-3-methylbutyrates and a pharmaceutically acceptable carrier. The pharmaceutically acceptable carrier may be present at a level of from about 0.1% to about 99%, more particularly from about 0.1% to about 75%, and in certain embodiments, from about 0.1% to about 50% by weight of the composition.

Unit dosage forms (i.e., compositions containing an amount of calcium and/or magnesium hydroxy-3-methylbutyrates suitable for administration in one single dose, according to sound medical practice) preferably contain from about 730 mg (milligrams) to about 7,300 mg of calcium hydroxy-3-methylbutyrate, corresponding to from about 100 mg to about 1,000 mg of calcium on an elemental basis, and from about 200 mg to about 2,000 mg magnesium hydroxy-3-methylbutyrate, corresponding to from about 19 mg to about 190 mg of magnesium on an elemental basis. The unit dosage forms in accordance with certain aspects of the present invention contain from about 730 mg to about 2,300 mg of calcium hydroxy-3-methylbutyrate and from 200 mg to about 600 mg of magnesium hydroxy-3-methylbutyrate.

Solid dosage forms include tablets, capsules, granules and bulk powders. Aside from the calcium and magnesium hydroxy-3-methylbutyrates, tablets may contain, as carriers, suitable binders, lubricants, diluents, disintegrating agents, coloring agents, flavoring agents, flow-inducing agents, melting agents, and mixtures thereof. Liquid oral dosage forms include aqueous solutions, emulsions, suspensions, solutions and/or suspensions reconstituted from non-effervescent granules and effervescent preparations reconstituted from effervescent granules. Such oral dosage forms may contain, as carriers (for example), suitable solvents, preservatives, emulsifying agents, suspending agents, diluents, sweeteners, melting agents, coloring agents, flavoring agents, and mixtures thereof.

Specific examples of pharmaceutically acceptable carriers and excipients that may be used to formulate oral dosage forms of the present invention are described in U.S. Pat. No. 3,903,297, Robert, issued Sep. 2, 1975, incorporated by reference herein. Techniques and compositions for making dosage forms useful in the methods of this invention are described in the following references, all incorporated by reference herein: Modern Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2d Edition. (1976).

The present invention also provides methods for the administration of a calcium and/or magnesium supplement to a human or other animal subject in need thereof, comprising administering to said subject a safe and effective amount of the calcium and/or magnesium hydroxy-3-methylbutyrates of this invention. Preferably, from about 730 mg to about 2,300 mg of calcium hydroxy-3-methylbutyrate is administered per day. “Administering” refers to any method which, in sound medical practice, delivers the calcium and/or magnesium hydroxy-3-methylbutyrates used in this invention to the subject to be treated in such a manner so as to be effective in the treatment or prevention of calcium or magnesium deficiency pathologies.

The calcium and magnesium hydroxy-3-methylbutyrates of this invention may be used to replace or supplement other dietary sources of calcium for humans or other animals. The supplements may be used to assure adequate intake of calcium and magnesium for metabolic needs, or to prevent or treat certain disorders arising from a deficiency of these elements.

Calcium and magnesium hydroxy-3-methylbutyrates may be added to foods or beverages in amounts that increase mineral absorption but that do not adversely affect the flavor or texture of the food. Suitable ranges for the amount of these additives can be readily determined by one skilled in the art.

The calcium and magnesium salts can be synthesized and isolated separately and subsequently blended to produce a composition with the desired ratio of calcium to magnesium. Alternatively, the precursor calcium and magnesium compounds (i.e. calcium and magnesium carbonates or calcium and magnesium oxides or hydroxides) can be mixed in the desired ratio in a preliminary step and co-dissolved in an aqueous solution of 3-hydroxy-3-methylbutyric acid to form a mixture of the calcium and magnesium hydroxy-3-methylbutyrates of this invention. In accordance with certain aspects of the invention calcium and magnesium are present at molar ratios of from about 2:1 to about 20:1 (Ca:Mg).

The following non-limiting examples illustrate the compositions, processes and uses of the present invention.

### EXAMPLE 1

74.1 grams (1.0 moles) of calcium hydroxide is added to approximately 400 ml of water with vigorous agitation, forming a slurry. 224 grams of 3-methyl-3-hydroxy-3-methylbutyric acid (1.90 moles) is introduced slowly. Following the addition of the acid, the mixture is heated to 70 degrees C. and stirred for 90 minutes, then allowed to cool to room temperature. Insoluble particles of excess lime are removed by filtration on a Buechner funnel through Whatman #4 (20 micron) filter paper. The filtrate, a clear, non-turbid liquid, is evaporated to dryness, producing crystalline calcium 3-hydroxy-3-methylbutyrate monohydrate in virtually quantitative yield.

In a separate vessel, 20.15 grams (0.50 moles) of magnesium oxide is added to approximately 200 ml of water with vigorous agitation, forming a slurry. 112 grams of 3-methyl-3-hydroxy-3-methylbutyric acid (0.95 moles) is introduced slowly. Following the addition of the acid, the mixture is heated to 70 degrees C. and stirred for three hours, then allowed to cool to room temperature. Insoluble particles of excess magnesium oxide are removed by filtration on a Buechner funnel through Whatman #4 (20 micron) filter paper. The filtrate, a clear, non-turbid liquid, is evaporated to dryness, producing crystalline magnesium 3-hydroxy-3-methylbutyrate in virtually quantitative yield.

The calcium and magnesium salts are ground together in a mortar and pestle to produce a mixture with a bulk density of 0.60 grams per cubic centimeter.

### EXAMPLE 2

74.1 grams (1.0 moles) of calcium hydroxide and 20.15 grams (0.50 moles) of magnesium oxide are added to 500 ml of water with vigorous agitation, forming a slurry. 336 grams of 3-methyl-3-hydroxy-3-methylbutyric acid (2.85 moles) is introduced slowly. Following the addition of the acid, the mixture is heated to 70 degrees C. and stirred for 90 minutes, then allowed to cool to room temperature. Insoluble particles of excess lime and magnesia are removed by filtration on a Buechner funnel through Whatman #4 (20 micron) filter paper. The filtrate, a clear, non-turbid liquid, is evaporated to dryness, producing an intimate mixture of crystalline calcium 3-hydroxy-3-methylbutyrate monohydrate and magnesium 3-hydroxy-3-methylbutyrate in virtually quantitative yield.

What is claimed is:

1. A method for preventing and/or treating a condition associated with calcium and/or magnesium deficiency in a human or other animal subject comprising orally administering a safe and effective amount of a mineral supplement comprising a salt selected from the group consisting of calcium 3-hydroxy-3-methylbutyrate, magnesium 3-hydroxy-3-methylbutyrate and mixtures thereof to a subject in need thereof.

2. The method of claim 1 wherein said mineral supplement comprises calcium and magnesium present at a molar ratio of from about 2:1 to about 20:1 (calcium:magnesium).

3. The method of claim 1 wherein said supplement further comprises a pharmaceutically acceptable carrier.

4. The method of claim 3 wherein said pharmaceutically acceptable carrier is present in said supplement at a concentration of from about 0.1% to about 75% by weight.

5. The method of claim 1 wherein said mineral supplement comprises calcium 3-hydroxy-3-methylbutyrate.

6. The method of claim 5 wherein said calcium 3-hydroxy-3-methylbutyrate is administered at a level of from about 100 to about 1000 mg (on an elemental calcium basis) per day.

7. The method of claim 1 wherein said mineral supplement comprises magnesium 3-hydroxy-3-methylbutyrate.

8. The method of claim 7 wherein said magnesium 3-hydroxy-3-methybutyrate is administered at a level of from about 19 to about 190 mg (on an elemental magnesium basis) per day.

9. The method of claim 1 wherein said supplement is in the form of an oral dosage form selected from the group consisting of solid dosage forms and liquid dosage forms.

10. The method of claim 1 wherein said condition is selected from the group consisting of osteoporosis, hypertension and bone loss.

11. The method of claim 1 wherein said mineral supplement is in the form of a beverage or food product.

12. A method for providing a nutritionally supplemental amount of calcium and magnesium to a human or other animal subject comprising orally administering to said subject a safe and effective amount of a mineral supplement comprising calcium 3-hydroxy-3-methylbutyrate and magnesium 3-hydroxy-3-methylbutyrate.

13. The method of claim 12 wherein said mineral supplement comprises calcium and magnesium present at a molar ratio of from about 2:1 to about 20:1 (calcium:magnesium).

14. The method of claim 12 wherein said supplement further comprises a pharmaceutically acceptable carrier.

15. The method of claim 14 wherein said pharmaceutically acceptable carrier is present in said supplement at a concentration of from about 0.1% to about 50% by weight.

16. The method of claim 15 wherein said mineral supplement is a unit dosage form.

17. The method of claim 16 wherein said unit dosage form comprises from about 730 mg to about 7,300 mg calcium 3-hydroxy-3-methylbutyrate and from about 200 mg to about 2,000 mg magnesium 3-hydroxy-3-methylbutyrate.

18. The method of claim 17 wherein said unit dosage form comprises from about 730 mg to about 2,300 mg calcium 3-hydroxy-3-methylbutyrate and from about 200 mg to about 600 mg magnesium 3-hydroxy-3-methylbutyrate.

19. A bioavailable and water-soluble composition for conjointly administering bioavailable calcium and magnesium in an oral form to a human patient or other animal in need thereof comprising (a) calcium in a water-soluble form, (b) magnesium in a water-soluble form, and (c) an edible organic acid component comprising 3-hydroxy-3-methylbutyric acid.

20. The composition of claim 19 wherein said composition is in the form of an oral dosage form selected from the group consisting of solid dosage forms and liquid dosage forms.

21. The composition of claim 19 wherein said composition comprises calcium and magnesium present at a molar ratio of from about 2:1 to about 20:1 (calcium:magnesium).

22. The composition of claim 19 wherein said composition further comprises a pharmaceutically acceptable carrier.

23. The composition of claim 22 wherein said pharmaceutically acceptable carrier is present in said composition at a concentration of from about 0.1% to about 75% by weight.

24. The composition of claim 19 wherein said composition is a unit dosage form comprising from about 730 mg to about 7,300 mg calcium 3-hydroxy-3-methylbutyrate and from about 200 mg to about 2,000 mg magnesium 3-hydroxy-3-methylbutyrate.

25. The composition of claim 19 wherein said composition is in the form of a beverage or food product.
